(19) United States
(12) Patent Application Publication
(10) Pub. No.: US 2006/0116646 A1
(43) Pub. Date: Jun. 1, 2006

(54) Bi-inoculator dual syringe clip

(76) Inventor: Jennifer J. Weiss, Kansas City, MO (US)

Correspondence Address: JAMES RAY & ASSOCIATES, 2640 Pitcairn Road, Monroeville, PA, 15146, US

(21) Appl. No.: 11/168,150

(22) Filed: Jun. 28, 2005

Related U.S. Application Data
(60) Provisional application No. 60/583,729, filed on Jun. 28, 2004.

(51) Int. Cl. A61M 5/00

(52) U.S. Cl. 604/191

(57) ABSTRACT

The bi-inoculator dual syringe clip of the invention is a unique piece of equipment that is a lightweight, convenient, sterile, and easy to use device that clips two existing syringes together which allows two inoculations to be combined into one step without having to combine the antibodies. The bi-inoculator dual syringe clip comprises a first and second half having at least a first and second pair of curved portions separated by a first and second spacer member. A hinged portion connects the first and second halves together such that the halves can be folded together causing the first pair of curved portions to mate with the second pair of curved portions to form a pair of tubular holding members capable of holding a syringe therein. The bi-inoculator dual syringe clip of the invention improves the method of administering inoculations tremendously and also improves patient care as well as reduces trauma.

### CROSS-REFERENCES TO RELATED APPLICATIONS

The present application is closely related to and claims benefit from U.S. Provisional Application Ser. No. 60/583,729 filed Jun. 28, 2004.

### FIELD OF THE INVENTION

The present invention relates in general to a medical device for the application of inoculations and, more particularly, to a clip for holding at least two syringes for the application of dual inoculations in a single step while avoiding cross-contamination of the antibodies.

### BACKGROUND OF THE INVENTION

For years, the medical industry has attempted to combine doses of antibodies into single dose inoculations. Although many combinations of antibodies have been successful, such as with the combination of Diphtheria, Tetanus, and Pertussis, it stands to reason that not all doses will be able to be combined into one single injection. As a result, infants and toddlers can receive four or more single shots during required immunizations. Adults, as well, often require multiple shots during immunization boosters. This can lead to a significant amount of pain, stress, and trauma for the patient, as well as, the parent and/or the person administering the shots.

As discussed in U.S. Pat. No. 3,552,394, a well-known phenomenon is the two-point threshold of pain or pressure. This phenomenon is defined as the distance at which the surface pain sensors of the human body cannot distinguish between single and multiple locations of pain. In other words, when applied to certain locations on the body, a person cannot distinguish between the applications of one or multiple needles when these needles are disposed comparably close with one another. U.S. Pat. No. 3,552,394 attempts to capitalize on this theory through the design of a dual hypodermic syringe device, which includes laterally spaced barrels, a needle associated with each of the barrels, and a plunger also associated with each of the barrels. The syringe device is made of plastic or glass, which is drilled to form the barrels. The barrels are capable of being filled with fluid and the needles and plungers are associated with these barrels. This device has several disadvantages. This device requires assembly of a plurality of separate parts making it difficult to use, it is costly to produce, requires sterilization before each use.

There is a need in the art for a dual syringe clip, which is lightweight, easy to use, convenient, and sterile. There is also a need in the art for a dual syringe clip, which securely holds the syringes in place during application. There is further a need in the art for a dual syringe clip that is inexpensive to produce and is disposable.

### OBJECTS OF THE INVENTION

It is therefore an object of the invention to produce a lightweight, low cost, and sterile medical device, which will improve the administration of immunizations and/or inoculations to patients.

It is yet another object of the invention to produce a dual syringe clip which will enable the administration of dual inoculations in a single step thus reducing the pain and trauma to the patients.

It is still another object of the invention to produce a dual syringe clip which will enable the administration of dual inoculations in a single step thus reducing the stress of the medical staff and/or parent or guardian of the patient associated with the application of multiple inoculations.

It is still yet another object of the invention to produce a dual syringe clip that will enable the administration of dual inoculations without combining the antibodies.

In addition to the various objects and advantages of the invention which have been described in some specific detail above it should be noted that various other objects and advantages of the present invention will become more readily apparent to those persons who are skilled in the relevant art from the following more detailed description, particularly, when such description is taken in conjunction with the attached drawing Figures and with the appended claims.

### SUMMARY OF THE INVENTION

Briefly, and in accordance with the forgoing objectives, the invention comprises a bi-inoculator dual syringe clip, which is a medical device that is designed to administer dual inoculations. This unique piece of equipment is a lightweight, convenient, sterile, and easy to use device that clips two existing syringes together, which allows two inoculations to be combined into one step without having to combine the antibodies. The bi-inoculator dual syringe clip of the present invention comprises a first half having at least a first pair of curved portions separated by a first spacer member and a second half, which is essentially symmetrical to the first half, having at least a second pair of curved portions separated by a second spacer member. A hinged portion connects the first and second halves together. The first and second halves are capable of being folded together along the hinged portion causing the first pair of curved portions to mate with the second pair of curved portions to form a pair of tubular holding members separated by the first and second spacer members. Each of the holding members is capable of holding a syringe therein. A means for joining the first and second halves together is also provided. The bi-inoculator dual syringe clip of the invention improves the method of administering inoculations tremendously and also improves patient care as well as reduces trauma.

### BRIEF DESCRIPTION OF THE DRAWINGS

FIG. 1 shows a top view of the bi-inoculator syringe clip of the invention.

FIG. 2 shows a bottom view of the bi-inoculator syringe clip of the invention.

FIG. 3 shows a top view of the bi-inoculator syringe clip of the invention in a partially open position.

FIG. 4 shows a top view of the bi-inoculator syringe clip of the invention in a fully open position.

FIG. 5 shows the bi-inoculator syringe clip of the invention holding a pair of syringes therein.

### DETAILED DESCRIPTION OF THE DRAWINGS

Before describing the invention in detail, the reader is advised that, for the sake of clarity and understanding, identical components having identical functions have been marked where possible with the same reference numerals in each of the Figures provided in this document.

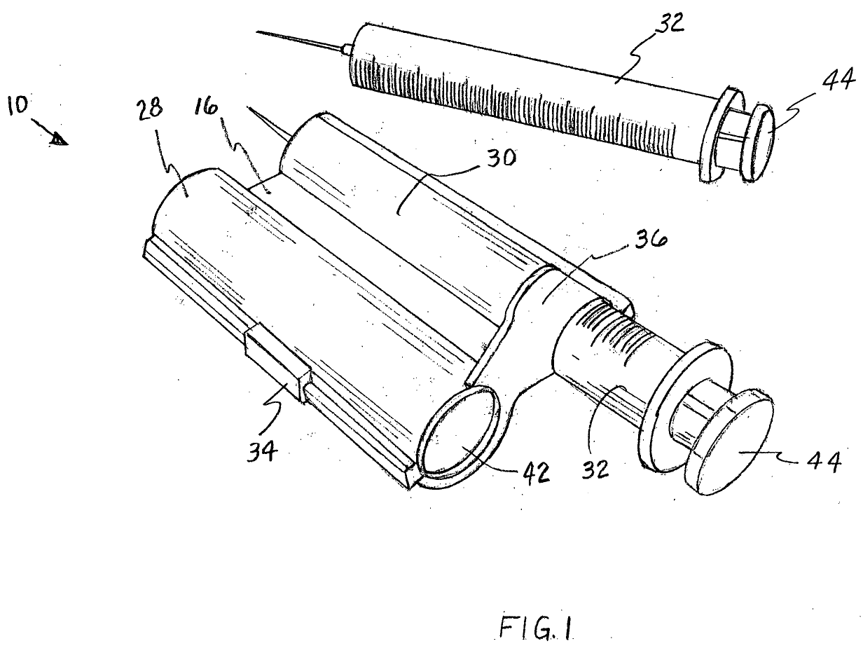

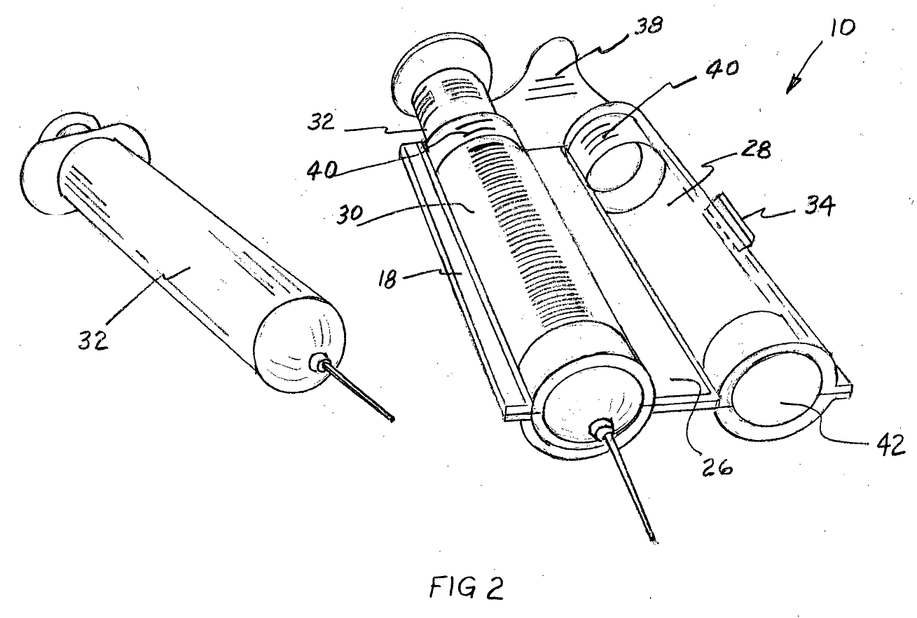

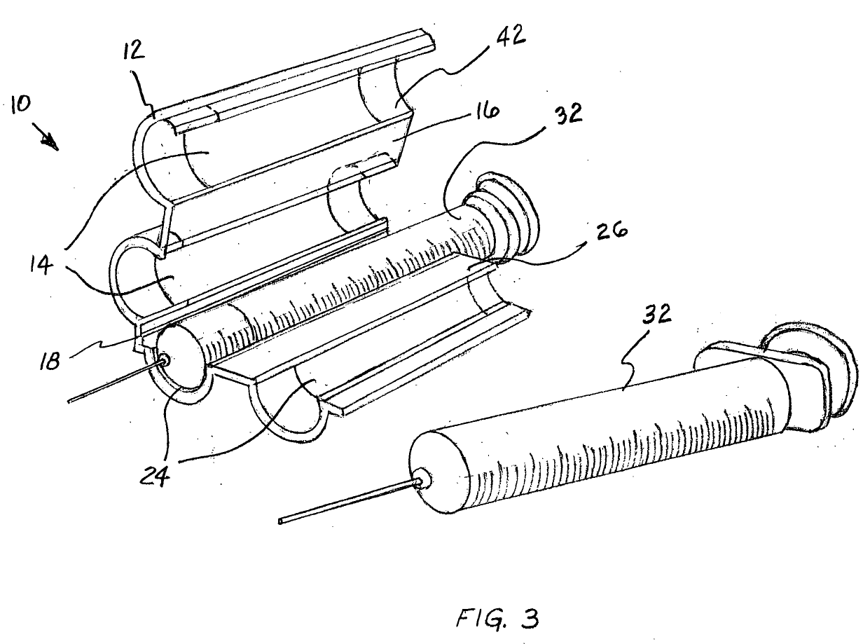

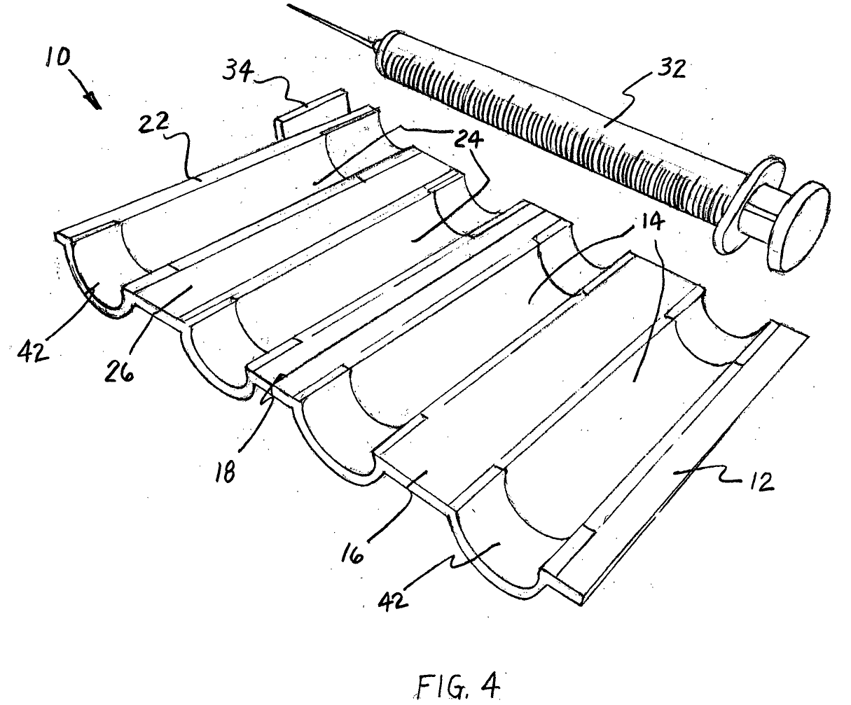

Referring now to FIGS. 1-5, there is shown the bi-inoculator dual syringe clip of the invention, generally illustrated as 10. As shown in FIGS. 3 and 4, the bio-inoculator dual syringe clip comprises a first half 12, having at least a first pair of curved portions 14 separated by a first spacer member 16 and a second half 22, which is essentially symmetrical to the first half 12, having at least a second pair of curved portions 24 separated by a second spacer member 26. A hinged portion 18 connects the first 12 and second 22 halves together. The first 12 and second 22 halves are capable of being folded together along the hinged portion 18 causing the first pair of curved portions 14 to mate with the second pair of curved portions 24 to form a pair of tubular holding members 28, 30 separated by the first 16 and second 26 spacer members. Each of the holding members 28, 30 is capable of holding a syringe 32 therein. A locking or fastening means 34 is provided for joining the first 12 and second 22 halves together.

The administration of the doses would be in the same manner as a single dose. The syringe clip 10 would be used long ways between the middle and pointer fingers. As illustrated in FIGS. 1, 2, and 5, at least one convenient finger support means 36 may be provided to assist the user in maintaining a firm grip and allow for proper maneuverability of the syringe clip 10 during application of the inoculations. The finger support means 36 extend outward from one or both of the first 12 and second 22 halves of the clip. An optional curved finger support design may also be provided. For added security during administration of doses and depression, ribbed portions 38 may be provided underneath the finger support means 36 to further assist the user with gripping of the syringe clip 10. Alternatively and/or additionally, ribbed portions 40 may also be provided on an outer portion of the first 12 and second 22 halves of the syringe clip 10.

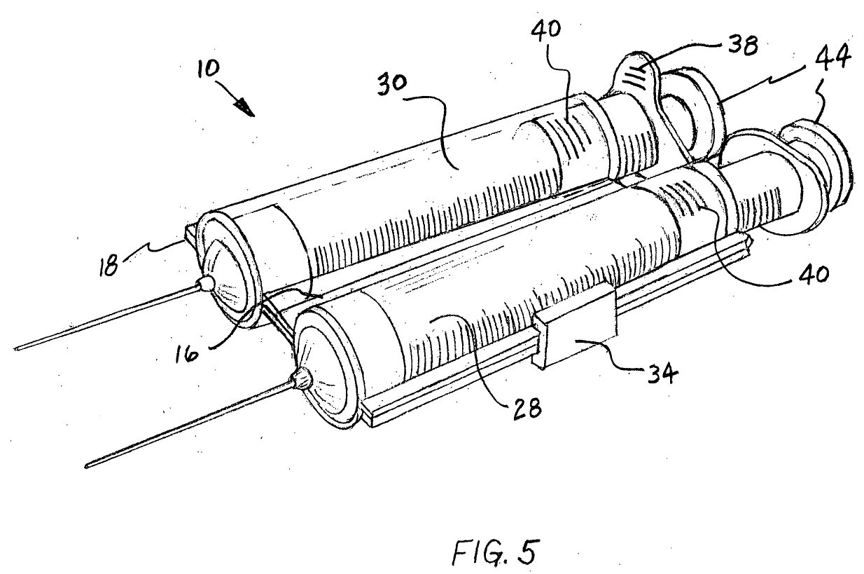

An optional plunger cover 46, as shown in FIG. 5, is available for depressing the plungers 44 of the syringes 32. This plunger cover 46 can be formed from any well-known flexible material. The cover 46 may not be needed, as most technicians will be able to use their thumb to depress both syringes simultaneously.

Gripping means 42 are positioned within the tubular holding members 28, 30 to hold the syringes 32 in place. The gripping means 42 can be any well-known gripping material such as a flexible plastic gripping compound or plastic grip liner or rubber material.

The syringe clip 10 can be formed from a single molded piece of material from any well-known material such as lightweight plastic or metal material.

As shown in FIGS. 1, 2, and 5, the doses in existing syringes 32 are separated in the middle by a solid piece (spacer member 16, 26) wide enough that the antibodies will not interfere with each other. The length of the clip 10 is long enough to secure the entire syringe 32 and keep it from moving, but not long enough to interfere with the visible sight of a fully depressed plunger 44. Doses are drawn into existing syringes 32 and then the clip 10 is put into place. The syringe lip would rest on the clip 10 for proper placement. Various sizes of the clip 10 can be manufactured to properly fit a variety of syringe types.

When used, the bi-inoculator reduces the pain, stress, and trauma of this much-needed procedure (for patient, nurse and parent when applicable). When the two syringes are depressed simultaneously, only one is felt. Preparation time for the bi-inoculator would take a little longer, as two doses would have to be drawn and then the syringes clipped together. The disposable clip could simply be discarded into the Bio Hazard container along with the syringes. Although using the bi-inoculator adds an extra step for the medical staff, it is well worth the benefits.

The bi-inoculator dual syringe clip of the present invention is not limited to embodiments wherein only two holding members for the application of two syringes are provided. The invention intends to include embodiments wherein the first half and second half of the clip include more than two curved portions separated by spacer members such that upon folding of the first and second halves together, the curved portions mate to form more than two tubular members capable of holding syringes therein.

The invention has been described in such full, clear, concise, and exact terms so as to enable any person skilled in the art to which it pertains to make and use the same. It should be understood that variations, modifications, equivalents and substitutions for components of the specifically described embodiments of the invention may be made by those skilled in the art without departing from the spirit and scope of the invention as set forth in the appended claims. Persons who possess such skill will also recognize that the foregoing description is merely illustrative and not intended to limit any of the ensuing claims to any particular narrow interpretation.

I claim:

1. A bi-inoculator dual syringe clip comprising:
(a) a first half having at least a first pair of curved portions separated by a first spacer member;
(b) a second half having at least a second pair of curved portions separated by a second spacer member;
(c) a hinged portion connecting said first and second halves together, said first and second halves capable of being folded together along said hinged portion causing said first pair of curved portions to mate with said second pair of curved portions to form a pair of tubular holding members separated by said first and second spacer members, each of said holding members capable of holding a syringe therein; and
(d) means for joining said first and second halves together.

2. A bi-inoculator dual syringe clip as recited in claim 1 including at least one finger support means extending from at least one of said first half and second half.

3. A bi-inoculator dual syringe clip as recited in claim 2 wherein each of said first half and second half includes a finger support means extending there from.

4. A bi-inoculator dual syringe clip as recited in claim 1 wherein said means for joining said first and second halves comprises a locking mechanism.

5. A bi-inoculator dual syringe clip as recited in claim 1 further including a gripping means positioned within said tubular holding members.

6. A bi-inoculator dual syringe clip as recited in claim 5 wherein said gripping means comprises one of a flexible plastic gripping compound and a plastic grip liner.

7. A bi-inoculator dual syringe clip as recited in claim 1 further including ribbed portions on an outer portion of said first and second halves to assist with gripping said clip.

8. A bi-inoculator dual syringe clip as recited in claim 3 further including ribbed portions positioned under said finger support means to assist with gripping said clip.

9. A bi-inoculator dual syringe clip as recited in claim 1 wherein said clip is formed from a single molded piece of material.

10. A bi-inoculator dual syringe clip as recited in claim 1 wherein said clip is formed from lightweight plastic.

11. A bi-inoculator dual syringe clip as recited in claim 1 wherein said holding members have a length which is long enough to secure the entire length of said syringes therein without interfering with sighting of a depressed plunger.

12. A bi-inoculator dual syringe clip as recited in claim 1 further including a plunger cover for covering and simultaneously depressing each syringe positioned within said holding members.

13. A bi-inoculator dual syringe clip as recited in claim 1 wherein said spacer members are of a sufficient width to avoid cross-contamination of the contents of said syringes during application of said contents to a patient.

14. A bi-inoculator dual syringe clip as recited in claim 1 wherein said spacer members are of a sufficient width such that a patient only feels a single sensation of pain during application of the contents of said syringes.

15. A bi-inoculator dual syringe clip as recited in claim 1 wherein said first half and said second half include more than two curved portions separated by spacer members such that upon folding of said first and second halves together, said curved portions mate to form more than two tubular members capable of holding syringes therein.
